(19) United States
(12) Patent Application Publication
(10) Pub. No.: US 2018/0321220 A1
(43) Pub. Date: Nov. 8, 2018

(54) UNIVERSAL BIOELECTROCHEMICAL METABOLIC FLUX MEASUREMENT SYSTEM AND METHODS OF MAKING AND USING THE SAME

(71) Applicant: Saint Louis University, St. Louis, MO (US)

(72) Inventors: Robert Louis ARECHEDERRA, Clayton, MO (US); Abdul WAHEED, Valley Park, MO (US); William S. SLY, St. Louis, MO (US)

(73) Assignee: Saint Louis University, St. Louis, MO (US)

(21) Appl. No.: 15/774,889

(22) PCT Filed: Nov. 8, 2016
(86) PCT No.: PCT/US16/60894

§ 371 Date: May 9, 2018

Related U.S. Application Data
(60) Provisional application No. 62/253,521, filed on Nov. 10, 2015.

(51) Int. Cl. G01N 33/50; G01N 27/02

(52) U.S. Cl. CPC G01N 33/5005; G01N 27/021

(57) ABSTRACT

A method for monitoring the metabolic state of an organism, cell, tissue, group of cells, organelles or organelle with or without a metabolic modulating agent or with or without a genetic alteration capable of modulating metabolism is disclosed. The biological material of interest is placed in a conductive solution in close proximity to a first electrode that is electrically coupled to a second electrode. A potential is applied to the electrodes sufficient enough to create a potential gradient between the two. If the biological material of interest is undergoing oxidation reactions, reduction reactions, or producing electrochemically active compounds as a result of metabolism, these will react at the first electrode, and in some cases, achieve direct electron transfer to the first electrode and generate a detectable electrical current. This current is directly proportional to the metabolic rate of the biological material of interest.

The present application claims benefit of priority to U.S. Provisional Application Ser. No. 62/253,521, filed Nov. 10, 2015, the entire contents of which are hereby incorporated by reference.

### BACKGROUND

### 1. Field

The present disclosure relates generally to the fields of cell biology, cell physiology, and biochemistry. More particularly, it concerns the methods and devices for measuring oxidoreductive metabolism in organelles, cells and whole organisms.

### 2. Description of Related Art

The efficient monitoring of metabolic activity, whether in organelles, cells, tissues, or whole organisms, is of increasing interest due to the necessity of biochemical energy production. Perturbation or modulation of any single step or pathway of the metabolic process can have dramatic cascading physiological effects that propagate all the way up to the whole organism level. This perturbation or modulation can be due to genetic alterations, inhibitory compounds, or activating compounds.

The alteration of the metabolic process can be due to genetic flaws or manipulations that affect enzymes or complexes that are involved in metabolic pathways. In some cases these genetic alterations are fatal to an organism, but in other cases, these types of diseases are treatable if they are diagnosed early enough. For example individuals with complex I and/or complex II deficiency may benefit from oral administration of riboflavin (Chinnery P F. Mitochondrial Disorders Overview, 2000 Jun. 8). It has been suggested that there are some diseases that are due to subtle changes in the genetic coding for components of the metabolic pathways. These subtleties in metabolism are difficult to trace back to the site of disruption of the metabolic process due to the plethora of physiological changes that occur when metabolism is even slightly altered.

Also, many external influences on the metabolic process exist. Different types of compounds can affect the metabolic pathways by targeting particular enzymes or complexes. In some cases, these compounds have become powerful pharmaceuticals that are used to treat diseases such as cancer, diabetes, and obesity. For example, many potent anesthetics such as propofol and lidocaine target the metabolism in nerve tissue to achieve their therapeutic effect. Unfortunately, due to the lack of understanding at the time these therapeutics were developed, unappreciated side effects were later discovered due off target interactions. For example, a potentially fatal side effect of propofol is heart failure. Propofol's mechanism of action is that it uncouples the respiratory chain of the nerve tissue mitochondria causing a large decrease in available ATP and reduced cofactors. However, propofol is not specific to nerve tissue mitochondria. It also affects other tissue mitochondria, including cardiac tissue which in many documented cases has led to cardiac arrest and heart failure (Oxford Journals Medicine & Health BJA: CEACCP Volume 13, Issue 6 Pp. 200-202).

Another infamous example is fen-phen, an anti-obesity drug developed by Wyeth that was subsequently removed from the market due to a very high incidence of cardiac effects. (Regulatory Toxicology and Pharmacology Volume 48, Issue 2, July 2007, Pages 115-117). Fen-phen's mode of action was intended to uncouple the respiratory chain of the muscle and fat tissue mitochondria in order to artificially increase metabolism and burn extra calories so that the subject would lose weight. Unfortunately, fen-phen also affected cardiac tissue in addition to other targeted tissues which lead to it being pulled from the market.

Clearly, the metabolic processes for energy production are important to many organisms. Even slight changes in one step of a pathway can have dramatic effects. To date, there is no universal, high-throughput method for directly monitoring metabolic flux of organelles, cells, tissues, and whole organisms. Such a method would be extremely powerful for determining on- and off-target drug candidate interactions, as well as aiding the understanding of fundamental disease mechanisms and downstream tissue and whole organism physiological effects. Current methods which involve cell death assays, pH monitoring, oxygen depletion, substrate production, or florescence techniques are not universal among different types of samples under study. In addition, they do not lend themselves to high throughput. Furthermore, because they rely on relatively indirect measurements of metabolic rate, they can yield spurious results. For example, pH monitoring has some utility, but there are many biochemical pathways and complex feedback mechanisms that affect pH which can complicate their interpretation. Thus, improved methods addressing these limitations remain in great need.

### SUMMARY

The disclosure relates to a method of measuring an oxidoreductive reaction in an organelle, cell or organism comprising (a) providing the organelle, cell or organism in a conductive solution comprising an electrolyte and (b) locating a first electrode in the conductive solution within about 2.0 mm of the organelle, cell or organism, wherein the organelle, cell or organism may or may not be in direct contact with the first electrode; (c) locating a second electrode in the conductive solution, wherein the organelle, cell or organism may or may not be in direct contact with the first electrode; (d) applying a potential to the first electrode, and an opposite potential to the second electrode, thereby generating a potential gradient; and (e) measuring electrical current across the first and second electrodes, wherein detection of the electrical current indicates the presence of an oxidoreductive reaction in the organelle, cell or organism, or the production of electrochemically active compounds by the organelle, cell or organism.

The cell may be is located in a tissue sample or tissue culture. The organelle may be a nucleolus, a nucleus, a ribosome, a vesicle, a rough endoplasmic reticulum, a Golgi apparatus, cytoskeleton, a smooth endoplasmic reticulum, a mitochondrion, a mitoplast, a vacuole, a chloroplast, a thylakoid, a lysosome, and a centriole. The organism may be a single-cell organism, a cell line, or embryo. The organism may be a multicellular organism, such as an invertebrate larva, invertabrate pupae, mature invertabrate, vertebrate in in all stages of development including just after embryonic stage.

The conductive solution may comprise metabolic substrates. The first electrode may be a working electrode and the second electrode may be a counter electrode. The conductive solution may be a buffered solution comprising DMSO. The conductive solution may be a hypotonic or hypertonic solution. The conductive solution may be an isotonic solution. The method organelle, cell or organism, the first and second electrodes, and the conductive solution may be disposed in a tissue culture dish, a well of a tissue culture tray, inserted into an organism, a screen printed electrode, in a test tube, or vial. The electrical current may be quantified.

The method may further comprise locating a third electrode in the conductive solution, the third electrode being a quasi-reference electrode. The organism may be rendered sufficiently permeable to allow compounds to taken up by the organism, such as where the organism is intact. Alternatively, the organism may be dissected.

The method further may comprise performing steps (a)-(e) a second time. The organelle, cell or organism may be subjected to a treatment between the first and second measuring steps. The treatment may comprise culturing of the organelle, cell or organism with a single component or multiple of the following: a toxin, a pesticide, a herbicide, an explosive, a solvent, an industrial chemical, a pollutant, a therapeutic small molecule, a biological agent, a genetic modifying agent, a radioactive compound, signaling cell compound, an organelle signaling compound, a redox compound, a therapeutic large molecule, a drug antibody conjugate, a nanomaterial, a polymer, a surfactant, an oligosaccharide, a saccharide, a fatty compound, a hormone, a cholesterol, a cytokine, a protein, a coenzyme, a vitamin, an antioxidant, a catalyst, a DNA section, an RNA section, an extract from another organism, an acid, a base, an isotopically enriched compound, an exposure to electromagnetic radiation from any portion of the electromagnetic spectrum or exposure to electromagnetic fields, an exposure to elevated or reduced temperatures, an exposure to elevated or reduced pressures, a gaseous compound.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1—Representative metabolic pathway for several substrates illustrating some key intermediates formed that can be used in other necessary biological processes. Abbreviations: ATP=Adenosine Triphosphate; NADH=Nicotinamide adenine dinucleotide (Reduced); COA=Coenzyme A; FADH2=Flavin adenine dinucleotide (Reduced); GTP=Guanosine triphosphate.

FIG. 2—Electron flux generated from mouse liver mitochondria suspension using pyruvate as a substrate with rotenone present was 1.435×10⁻⁴ micromoles of electrons with a standard deviation of 6.128×10⁻⁵ for 120 seconds of measurement.

FIG. 3—Electron flux generated from mouse liver mitochondria suspension using citrate as a substrate with rotenone present was 1.536×10⁻⁴ micromoles of electrons with a standard deviation of 1.571×10⁻⁵ for 120 seconds of measurement.

FIG. 4—Electron flux generated from whole drosophilae larvae using glucose as a substrate with rotenone present was 3.302×10⁻⁴ micromoles of electrons with a standard deviation of 1.087×10⁻⁴ for 120 seconds of measurement.

### DESCRIPTION OF ILLUSTRATIVE EMBODIMENTS

In the search of treatments for cancer, diabetes, asthma, obesity, and other debilitating diseases, many potential therapeutics have been found to negatively affect metabolic function, causing unwanted side effects. However, there are also numerous potential and current therapeutic agents that act by altering steps in the metabolic pathways and function in a beneficial manner, causing a desired physiological effect. As stated above, methods for monitoring the metabolic state of organelles, cells, tissues, and whole organisms, including in the presence of a potential metabolic modulating agent, are of great interest.

The inventors now disclose assays that permit the study cellular metabolism by presenting flexible, high-throughput and universal methods for such. In particular, the present assays permit examination of cellular metabolism by virtue of electron production, and in specific applications, assessing drug/compound-induced and genetic mutation-induced changes in mitochondrial and metabolic pathway function. The assays can functionally scale from an organelle to an entire organism. As disclosed herein, building on previous work using bio-electrochemical techniques on whole viable organelles such as mitochondria to generate electricity, the inventors have developed a scalable method for measuring metabolism from organelles up to whole organisms. This approach permits discovery of modulators such as drugs, or genetic alterations that affect metabolism of an organelles, cells, tissues, or organisms. An important distinction is that immobilization matrix for the sample is not required, and the assay is performed in solution, which together allow for samples to be tested in a simpler and more high-throughput fashion.

The methods in general provide one electrode in close proximity to a target organelle, cell, tissue, or whole organism that is electrically coupled to a second electrode of sufficient differential polarity in a circuit. The electrode within close proximity to the target is contacted with an conductive aqueous carrier, as is the second electrode, such carrier which may further contain a further fluid, gas, solid, or mixture thereof and contains an electrolyte. This aqueous carrier may or may not contain a potential metabolic modulating agent, and also may or may not contain a metabolic substrate depending on the nature of the experiment. During metabolism, substrates either already within the target or contained in the carrier are reacted stepwise through the metabolic pathway to form anionic products, cationic products, reduced products, partially oxidized products, and/or electrochemically active compounds from the substrate or part of the substrate that is released into the aqueous carrier-containing electrolyte, thereby providing a current between the electrodes. A metabolic flux data set may be obtained during the reaction/reactions using one or both of those electrodes, and may be compared to a control metabolic flux data set obtained under the same conditions in the absence of the potential metabolic modulating agent or genetic alteration, thereby determining the metabolic state in the presence of the potential metabolic modulating agent or genetic alteration.

### A. METHOD OVERVIEW AND COMPONENTS OF THE SYSTEM

The methods comprise providing a first electrode in close proximity to a target organelle, cell, tissue, or whole organism that is electrically coupled to a second electrode of sufficient differential polarity in a circuit. The electrode within close proximity to the target is contacted with an conductive solution or carrier that contains an electrolyte, and may further contain fluid, gas, solid, or mixture thereof This aqueous carrier may or may not contain an amount of a potential metabolic modulating agent, and also may or may not contain an amount of a metabolic substrate depending on the nature of the experiment, as discussed further below. The entire system is disposed in a container that permits retention of the carrier, and disposition of the target and both electrodes.

During metabolism, substrates either already within the target or contained in the carrier are reacted stepwise through the metabolic pathway to form anionic products, cationic products, reduced products, partially oxidized products, or electrochemically active compounds from the substrate or part of the substrate that is released into the aqueous carrier-containing electrolyte to thereby provide a current between the electrodes. A metabolic flux data set is obtained during the reaction/reactions using one or both of those electrodes, and may be compared to a control metabolic flux data set obtained under the same conditions in the absence of the potential metabolic modulating agent or genetic alteration, thereby determining the metabolic state in the presence of the potential metabolic modulating agent or genetic alteration. Thus, the system may contain a device for detecting, and optionally quantifying, the electrical current.

1. Electrodes

The electrode can include a material selected from carbon-based material, a metallic conductor, a semiconductor, a metal oxide, a modified conductor, and combinations thereof. The electrode including a carbon-based material can be from the group consisting of carbon cloth, carbon paper, carbon screen printed electrode, carbon black, carbon powder, carbon fiber, single-walled carbon nanotube, double-walled carbon nanotube, multi-walled carbon nanotube, carbon nanotube array, diamond-coated conductor, glass carbon, mesoporous carbon, graphite, uncompressed graphite worms, delaminated purified flake graphite, high performance graphite, highly ordered pyrolytic graphite, polycrystalline graphite, amorphous carbon, an allotrope of carbon or modified allotrope of carbon, an organic conductive polymer, a redox polymer, a polymer composite, and a combination thereof.

2. Conductive Solution

The conductive solution of the present methods is an aqueous solution comprising an electrolyte. The electrolyte may be a salt such as but not limited to sodium chloride or other ionic compound or compounds capable of transferring charge. The solution may also comprise a variety of other agents, such as but not limited to growth factors, electrochemical reactive substances, energy sources, labels, cofactors, enzymes, solvents, co-solvents, substrates, buffers, and metabolic modulating agents.

3. Target Biological Materials

Target biological materials for assessing under the disclosure methods include subcellular organelles, single cells, multi-cellular structures (tissues), and organisms (single- and multi-celled). The materials may be “normal” or “healthy,” or they may be “abnormal” or “unhealthy,” meaning that the cells may be affected by genetic alterations that induce diseases states, or may be affected by epigenetic changes resulting in disease, such as infection, autoimmunity, or inflammation. The cells may be mammalian (e.g., human), invetebrate, protozoan, bacterial, plant or fungal. The cells may be cancerous, benign hyperproliferative or virally-infected.

An organelle, in cell biology and herein, is a specialized subunit within a cell that has a specific function that is usually separately enclosed within its own lipid bilayer or monolayer, and is typically within the cytoplasm of a cell. Major animal cell organelles and cellular structures include: (1) a nucleolus, (2) a nucleus, (3) a ribosome, (4) a vesicle, (5) a rough endoplasmic reticulum, (6) a Golgi apparatus, (7) cytoskeleton, (8) a smooth endoplasmic reticulum, (9) a mitochondrion, (10) a mitoplast, (11) a vacuole, (12) a lysosome, and (13) a centriole. Plant organelles include chloroplasts and thylakoids.

A cell in cell biology and herein, is a specialized singular unit that contains organelles, and is considered the finite unit of life. A tissue is defined herein as a specialized multi-cell unit of an organism that can perform one or more functions for the organism. An organism defined here as a specialized singular entity that contains multiple defined tissue types organized into individual organs, and can perform many higher level functions as a sum of its organs and tissues.

4. Ancillary Agents

In addition to measuring the oxidoreductive actions of the target alone, it may be useful to test ancillary reagents for their effects on cellular metabolic activity. For example, environmental agents such as toxins, pesticides, herbicides, explosives, solvents, industrial chemicals, pollutants may be tested. Alternatively, therapeutic drugs and biological agents can be tested. Biological agents, included but not limited to cytokines, chemokines, antibodies, cell growth/inhibitory factors, neurotransmitters, hormones, enzymes, cell signaling agents, cofactors, cell waste products, metabolic products, antibiotics, inhibitory compounds, activating compounds, proteins, saccharides, oligosaccharides and pheromones.

Another type of ancillary agent is one that permits a more facile detection. These include but are not limited to compounds or electroactive polymers that can act as a mediator in the electron or charge transfer reactions. These could also take the form of a material that helps shuttle another ancillary agent such as but not limited to a surfactant or biologically tagged material to allow uptake. This also includes but is not limited to systems where multiple methods and chemicals are used simultaneously such as but not limited to luciferin luciferase for florescence detection.

5. Electrical Current Detection Devices

The electrical current detection devices can include a potentiostat, galvanostat, amperostat, or combinations thereof. These can be coupled together and used with common reference and working electrodes or these can be used as individual devices each utilizing its own set of electrodes. These devices are scalable and one main system could host numerous individual channels or shared channels that could operate simultaneously.

### B. METHODS

A flexible, rapid, universal and high-throughput method for monitoring the metabolic state of an organelle, cell, tissue, or whole organism in the presence of a potential metabolic pathway modulating agent is also contemplated.

A screening assay for drugs or other modulators is contemplated. One embodiment contemplates a flexible assay for isolated organelles, cells, tissues, and whole organisms for screening potentially active compounds for causing metabolic dysfunction, which is currently done by lengthy and indirect measurements further down the drug development pathway; this assay permits one to test drug or metabolic pathway active compound candidates for drug induced toxicity at an earlier stage yielding more comprehensive data with less expense.

A second embodiment contemplates a quantitative and comprehensive determination of the effects of a drug or active compound on metabolism at the organelle level, the cell level, the tissue level, and the whole organism level allowing for the direct determination of the mode of action, the off target interactions, the physiological effects on the metabolism of individual cell types/tissues, and the also the final metabolic effect on the entire organism. In addition, a contemplated method permits synergistic compounds to be examined, where one compound alone may or may not have an effect, but when used in addition to another compound provides an enhanced effect or entirely different effect, or a return to normal function.

Another contemplated method permits therapeutic compounds to be examined, where a genetic alteration affects the metabolic process but with the addition of the therapeutic the metabolic pathways return to a closer to normal function. In certain embodiments, the present disclosure can also be used in connection with water treatment and testing as well as treatment of and testing for biological agents including but not limited to pesticides, herbicides, antibiotics, hormones, poisons, warfare agents, and environmental contaminants that affect one or more steps in the metabolic process.

In general, all of the foregoing contemplated methods comprise the steps of providing at a first electrode within close proximity to the target material that is electrically coupled to a second electrode of sufficient differential polarity in a circuit. The electrode that is within close proximity of the target material is contacted with an aqueous carrier that contains an electrolyte, may or may not contain an effective amount of a potential metabolic modulating agent, may or may not contain an effective amount of a metabolic substrate, and can optionally contain a further fluid, gas, solid, or mixture thereof. During metabolism, substrates either already within the target material or contained in the carrier are reacted stepwise through the metabolic pathway to form anionic products, cationic products, reduced products, partially oxidized products, or electrochemically active compounds from the substrate or part of the substrate that is released into the aqueous carrier-containing electrolyte, thereby providing current at the second electrode when the circuit is closed. A metabolic flux data set is obtained during the reaction/reactions using one or both of those electrodes to detect, and may further be compared to a control metabolic flux data set obtained under the same conditions in the absence of the potential metabolic modulating agent or genetic alteration, thereby determining the metabolic state in the presence of the potential metabolic modulating agent or genetic alteration.

Application to drug screening using isolated mitochondria. In accordance with the above method, an illustrative approach has been developed for directly assaying metabolic activity as a function of metabolic substrate to determine drug toxicity. By adding a suspension of mitochondria to the aqueous carrier contacting the carbon electrode surface, electrons can be intercepted from Complex IV in the electron transport chain before they can reduce oxygen. The intercepted electrons are rerouted, so that oxygen reduction can occur at a separate electrode, the counter electrode. This permits the direct measurement of electrical current and potential of the mitochondria during their metabolism of substrates like pyruvate, fatty acids, amino acids, and Kreb's cycle intermediates as a measure of metabolic flux when there are different concentrations of drug compound present. Mitochondria from animals, yeast/fungi and plants can be used, such as those from a mouse, rat, potato, or yeast. This technique provides for the development of high throughput mitochondrial drug candidate screening, as well as other applications where the quantitative study of mitochondrial activity is of interest.

Application to whole cells or organisms. Another illustrative approach in accordance with the above method has been developed for assaying metabolic activity as a function of metabolic substrate to determine drug toxicity with whole organisms. By adding an intact Drosophila larvae in contact with the aqueous carrier and within close proximity to the carbon electrode surface electrochemically active compounds generated during metabolic process of the organism can be measured. The rate of metabolism will be proportional to the current measured.

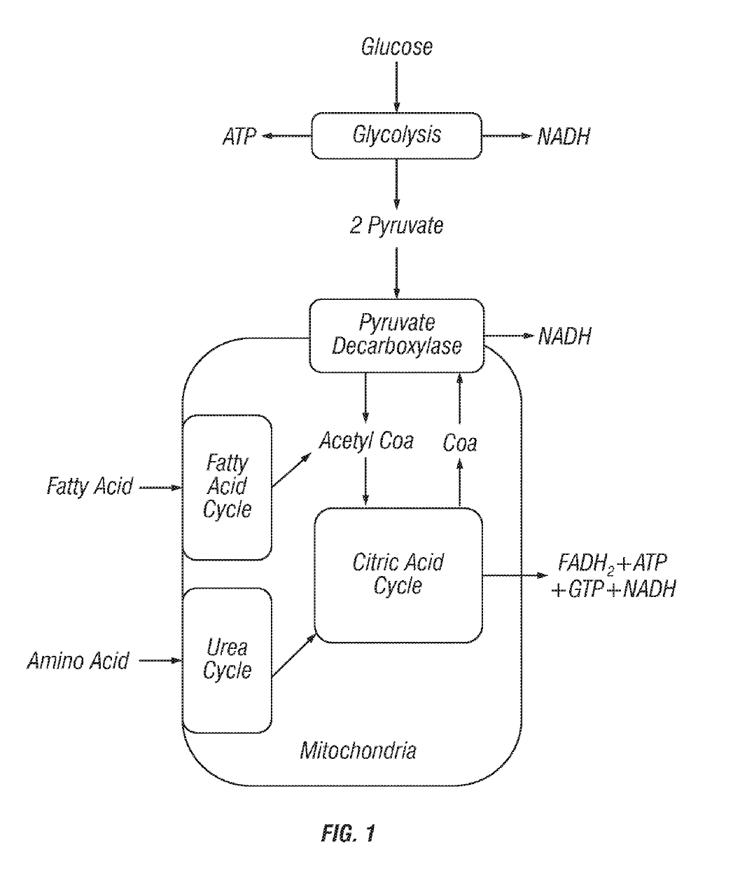

Referring to FIG. 1, a dominant role for the metabolic pathway is the production of ATP, NADH, NADPH, Acetyl-COA, as well as other more subtle but necessary product to be used in other cell and organelle functions. These include citric acid cycle intermediates, amino acids, fatty acids, and reduced compounds. This is done by oxidizing the major products of glucose, pyruvate, and NADH, which are produced in the cytosol, as well as fatty acids, and amino acids. This process of cellular respiration is known to be aerobic respiration which is dependent on the presence of oxygen. When oxygen is limited, the glycolytic products will be metabolized by anaerobic respiration, a process that is independent of the mitochondria. The production of ATP from glucose has an approximately 13-fold higher yield during aerobic respiration compared to anaerobic respiration. In comparison, the metabolism of a simple C10 fatty acid which is close in molecular weight to glucose will net 64 ATP which is 2-fold higher than the aerobic glucose.

Embedded in the inner membrane are proteins and complexes of molecules that are involved in the process called electron transport. The electron transport system (ETS), as it is called, accepts energy from carriers in the matrix and stores it to a form that can be used to phosphorylate ADP. Two energy carriers are known to donate energy to the ETS, namely nicotine adenine dinucleotide (NAD) and flavin adenine dinucleotide (FAD). Reduced NAD carries energy to complex I (NADH-Coenzyme Q Reductase) of the electron transport chain. PAD is a bound part of the succinate dehydrogenase complex (complex II). It is reduced when the substrate succinate binds the complex. When NADH binds to complex, it binds to a prosthetic group called Favin mononucleotide (FMN), and is immediately reoxidized to NAD. NAD is “recycled,” acting as an energy shuttle. In addition to the recycling FMN receives the hydrogen from the NADH and two electrons. It also picks up a proton from the matrix. In this reduced form, it passes the electrons to iron-sulfur clusters that are part of the complex, and forces two protons into the inter-membrane space. Electrons pass from complex I to a carrier (Coenzyme Q) embedded by itself in the membrane. From Coenzyme Q electrons are passed to a complex III which is associated with another proton translocation event. From Complex III the pathway is to cytochrome c then to a Complex IV (cytochrome oxidase complex). More protons are translocated by Complex IV, and it is at this site that oxygen binds, along with protons, and using the electron pair and remaining free energy, oxygen is reduced to water.

Since molecular oxygen is diatomic, it actually takes two electron pairs and two cytochrome oxidase complexes to complete the reaction sequence for the reduction of oxygen. This last step in electron transport serves the critical function of removing electrons from the system so that electron transport can operate continuously. The reduction of oxygen is not an end in itself. Oxygen serves as an electron acceptor, clearing the way for carriers in the sequence to be reoxidized so that electron transport can continue. Electron transport inhibitors act by binding one or more electron carriers, preventing electron transport directly. Changes in the rate of dissipation of the chemiosmotic gradient have no effect on the rate of electron transport with such inhibition. In fact, if electron transport is blocked, the chemiosmotic gradient cannot be maintained. No matter what substrate is used to fuel electron transport, only two entry points into the electron transport system are known to be used by mitochondria. A consequence of having separate pathways for entry of electrons is that an ETS inhibitor can affect one part of a pathway without interfering with another part. Respiration can still occur depending on choice of substrate. Some inhibitors may completely block electron transport by irreversibly binding to a binding site.

Each pyruvate molecule produced by glycolysis is actively transported across the inner mitochondrial membrane, and into the matrix where it is oxidized and combined with coenzyme A to form CO₂, acetyl-CoA, and NADH. The acetyl CoA is the primary substrate to enter the citric acid cycle, also known as the tricarboxylic acid (TCA) cycle or Krebs cycle. The enzymes of the citric acid cycle are located in the mitochondrial matrix, with the exception of succinate dehydrogenase, which is bound to the inner mitochondrial membrane as part of Complex II. The citric acid cycle oxidizes the acetyl CoA to carbon dioxide, and, in the process, produces reduced cofactors (three molecules of NADH and one molecule of FADH) that are a source of electrons for the electron transport chain, and a molecule of GTP (that is readily converted to an ATP). The redox energy from NADH and FADH₂ is transferred to oxygen (O₂) in several steps via the electron transport chain. These energy-rich molecules are produced within the matrix via the citric acid cycle but are also produced in the cytoplasm by glycolysis. Transport equivalents between the cytoplasm and the mitochondria can be imported/exported via the malate-aspartate shuttle system of antiporter proteins or feed into the electron transport chain using a glycerol phosphate shuttle. Protein complexes in the inner membrane (NADH dehydrogenase, cytochrome c reductase, and cytochrome c oxidase) perform the transfer and the incremental release of energy is used to pump protons (H⁺) into the inter-membrane space. This process is efficient, but a small percentage of electrons may prematurely reduce oxygen, forming reactive oxygen species such as superoxide. This can cause oxidative stress in the mitochondria and may contribute to the decline in mitochondrial function associated with the aging process. As the proton concentration increases in the inter-membrane space, a strong electrochemical gradient is established across the inner membrane. The protons can return to the matrix through the ATP synthase complex, and their potential energy is used to synthesize ATP from ADP and inorganic phosphate.

The present disclosure is generally directed to a method for monitoring perturbations in metabolism of organelles, cells, tissues, and whole organisms in response to a potential modulating agent such as an inhibitor, activator, uncoupler, pathway sidestepping, genetic alteration, or enhancement agent (collectively referend to as a modulator). The readout is an alteration in electrons generated, When a potential differential is present, whether it is created electronically or electrochemically between at least two electrodes in contact with an electrolyte medium the electrode that is within close proximity of the sample/subject the action of metabolism will generate one of or several anionic products, cationic products, reduced products, partially oxidized products, or electrochemically active compounds from the substrate or part of the substrate that is released into the aqueous carrier-containing electrolyte, thereby creating a current flow between the two electrodes. In the case of the sample being mitochondria, direct electron transfer to the electrode is possible but not necessary. In other sample types such as cells, tissues and whole organism in many cases it is the electrochemically active compounds that are either near the sample's surface or capable of being transported or diffused close enough to the electrode to achieve a current producing electrochemical reaction. The electrochemically active compounds that are being measured are either or both created directly by the metabolic process such as but not limited to NAD/NADH or from a downstream reaction pathway that was initiated by the pool of compounds that are created directly from metabolism such as but not limited to NAD/NADH.

Metabolic pathways are a series of chemical reactions that occur within a cell. In each pathway a principal chemical is modified by a series of chemical reactions. In many cases these reactions are enzymatically catalyzed. Because of the many chemicals known as metabolites that may be involved and each metabolite in many cases is not unique to only one pathway due to the numerous but distinct pathways in the cell, the term metabolic network is used. All of these pathways and networks work together to maintain the homeostasis of an organism. These pathways generally react to stimuli whether it be internal or external to adjust and maintain the homeostasis typically either directly or by feedback loops that can for example be as simple as a buildup or depletion of a substrate. While the equilibrium of a reaction is typically favored in a particular direction, in many chemical reactions within the cell they are reversible to a particular degree in order to aid with homeostasis.

Prior art has shown that immobilized mitochondria can yield different potentials and currents in response to substrate choice and inhibitor present. This uniqueness of current, potential, and metabolic energy conversion for each individual inhibitor yields a wealth of information that would be difficult and time consuming to acquire by traditional mitochondrial assay techniques. For example, most oxygen consumption experiments are on the order of hours of measurement time per sample. Because of the analytical nature of electrochemical measurements, very minute real time changes of metabolism in response to therapeutic levels of drug concentration are possible. This permits large chemical libraries to be tested at many different concentrations to determine if a potential modulating agent has an effect on metabolism of a particular substrate, if it does so at a therapeutic concentration, and what complex or enzyme the compound targets. This data is a powerful tool that can then be used to focus strictly on compounds that show the ability to efficiently target a particular aspect of the metabolic network to treat a particular disease by tuning the metabolic network or substrate pathway.

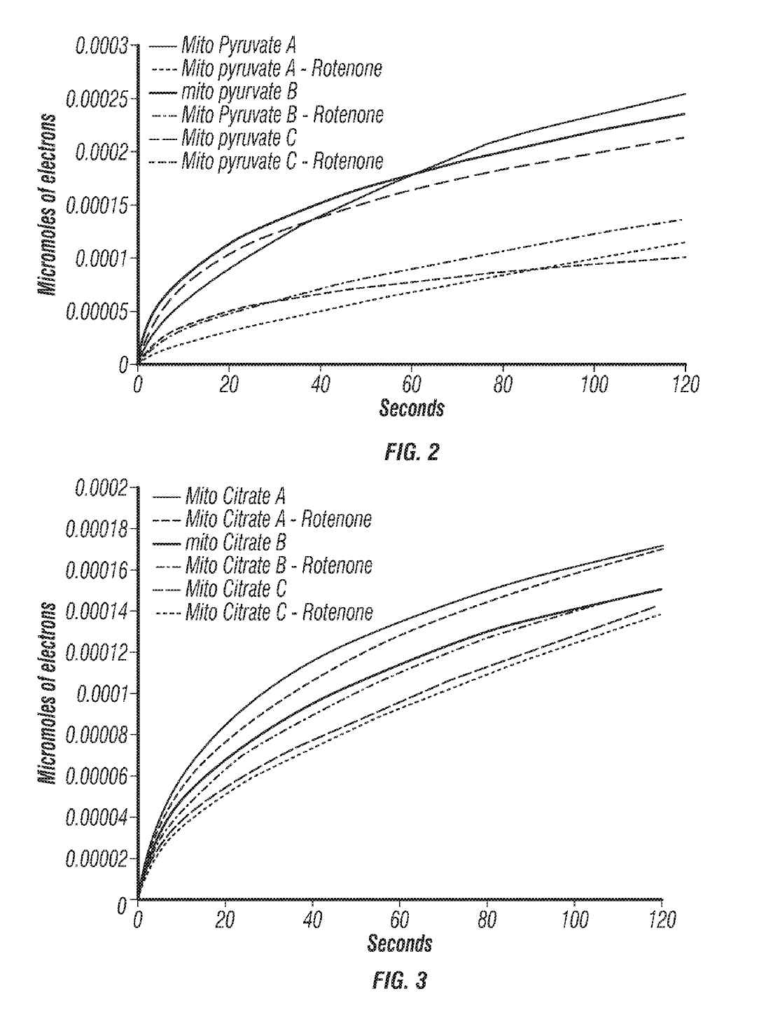

Monitoring Metabolic Flux With Respect to Classical Inhibitors. It was found that the attenuation of electron flux (which constitutes part of the metabolic flux and is directly relatable to oxygen consumption or substrate turnover) generated from mouse liver mitochondria was unique for each inhibitor or mitochondrial active compound with respect to the inhibitors or compounds target as shown in FIG. 2. In addition, it was also found that changing the metabolic substrate, such as pyruvate to a citric acid cycle substrate such as citrate, also gave unique attenuation for some of the inhibitors as shown in FIG. 3.

Unique electron flux profiles were observed for each combination of inhibitor and metabolic substrate. Because current is a measurement of the rate of electron flow, and oxygen reduction is a 4-electron process, the Oxygen consumption rate and the substrate turnover rate through the pathway is directly related. For example, diazoxide (an inhibitor of Complex II) only demonstrates partial inhibition with pyruvate (Dröse et al., Biochim. Biophys. Acta 1790, 558-565, 2009) that can be correlated with the partial oxidation of pyruvate in the citric acid cycle because the first one-half of the cycle is still active.

Both rotenone and antimycin demonstrated such strong inhibitory responses. From a fundamental point of view, if electron transport chain Complex I or Complex III are non-functional, the path of the electrons to the electrode is completely blocked. If Complex I is completely inhibited, no further metabolic reactions occur, resulting in no electrons being transported through the electron transport chain. If Complex III is inhibited, electrons cannot pass beyond that point resulting in the same effect. The only difference between the effect on the mitochondria is that, when Complex III is inhibited, the mitochondria can metabolize until all of their cofactors such as NAD+ and ubiquinone are reduced but, when Complex I is inhibited, the coenzymes are all fully oxidized. A very similar situation exists for cells, tissues, and whole organisms. However, these also have more pathways available such as but not limited to glycolysis which adds to the substrates that can be used to probe the metabolic network in order to find the target or targets of the modulators.

### C. KITS

In some embodiments, contemplated are kits comprising electrodes, conductive solutions (or components for making the same), and suitable containers for performing the assays described herein. In some embodiments, these kits contain controls and/or standards. In one embodiment these kits could be designed to allow for third party measurement devices to be coupled to the electrochemical cell. In another embodiment pre-made disposable electrodes and test samples could be provided either separately or together. These samples could be prepared tissue sections, cells, isolated organelles, or whole organisms that are ready to test. Another embodiment of a kit would be a library or experimental matrix of prepared substrates, metabolic modulating agents, solution components, buffers, electrolytes, other compounds, indicators, fluorescing agents, signaling agents, biologically active compounds or combinations thereof. Another embodiment would be instruments for preparing samples to be tested such as but not limited to instrumentation to allow for tissue biopsy, organelle preparation, cell harvesting, cell growth, or whole organism management.

### D. ABBREVIATIONS

CoQ=Coenzyme Q

ADP=Adenosine Diphosphate

ATP=Adenosine Triphosphate

NADH=Nicotinamide adenine dinucleotide (Reduced)

NAD=Nicotinamide adenine dinucleotide

CoA=Coenzyme A

Acetyl-COA=Acetylated Coenzyme A

FADH2=Flavin adenine dinucleotide (Reduced)

FAD=Flavin adenine dinucleotide

GTP=Guanosine triphosphate

GDP=Guanosine triphosphate

ETC=Electron transport system

TCA=Tricarboxylic acid

### E. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Materials. Sodium phosphate monobasic (Sigma), sodium phosphate dibasic (Sigma), sodium chloride (Sigma), sodium pyruvate (Sigma), citric acid (Sigma), rotenone (Sigma), adenosine diphosphate (Sigma), sucrose (Sigma), Tris-EDTA (Sigma), Protease inhibitor cocktail (Sigma), deionized water (in house), and dimethylsulfoxide (Sigma) were used as received. All wild-type Drosophila larvae were hatched and grown on standard cornmeal-agar-molasses-yeast food at room temperature. Individual larvae were selected used for metabolic flux analysis. Mitochondria were isolated from normal mice liver. All steps were carried out at 4° C. Tissues were kept on ice. The liver tissue was homogenized in 3 volumes of Buffer A (0.33 M sucrose, 1.33 m M Tris-EDTA, pH 7.4 buffer containing protease inhibitors) using potter Dounce homogenizer. Once homogenized two more volumes of Buffer A were added and mixed before centrifugation at 1000 Xg for 10-15 mins. The supernatant was then recovered and centrifuged 2 times more to remove impurities. Finally, the supernatant was recovered and centrifuged at 15,000 Xg for 20 mins. The mitochondrial pellet recovered from the bottom of the centrifuge tube and suspended at a protein concentration of 35 mg/ml in phosphate buffered saline at pH 7.5. Once the extraction was complete the mitochondria was stored at −80° C. in small aliquots for further use.

Metabolic flux analysis. The electrochemical test setup consisted of a USTAT 200 Bipotentiostat coupled to a Dropsens SPE 110 screen printed electrode chip that included a carbon working electrode, a carbon counter electrode, and a silver reference electrode. The working substrate solution consisted of pH 7.40 10 mM phosphate buffer, 137 mM sodium chloride, 10 mM metabolic substrate, and 1 mg/ml adenosine diphosphate. The inhibitor solution consisted of 1 mg/ml rotenone in dimethyl sulfoxide. For uninhibited experiments pure dimethyl sulfoxide was used to maintain consistency. To the electrode 35 μl of substrate solution was dropped on top of the 3 electrode working area of the chip ensuring that all of the electrode surfaces were thoroughly wetted. To this drop, 1 μl of dimethyl sulfoxide was added that may or may not contain inhibitor. When the experiment was ready to begin the sample was added to the electrode. For mitochondrial suspension, 15 μL of the suspension was added to the working area of the chip. For the larvae, one larvae was placed into the drop of solution on to the working electrode. Amperometry was then performed with an applied potential of 600 mV with respect to the reference electrode for 120 seconds.

### Example 2—Results

It was found that the electron flux (which constitutes part of the metabolic flux and is directly relatable to oxygen consumption or substrate turnover) generated from mouse liver mitochondria suspension using pyruvate as a substrate without inhibitor present was 2.092×10⁻⁴ micromoles of electrons with a standard deviation of 6.332×10⁻⁵ for 120 seconds of measurement (FIG. 2). It was found that the electron flux generated from mouse liver mitochondria suspension using citrate as a substrate without inhibitor present was 1.554×10⁻⁴ micromoles of electrons with a standard deviation of 1.485×10⁻⁵ for 120 seconds of measurement (FIG. 3).

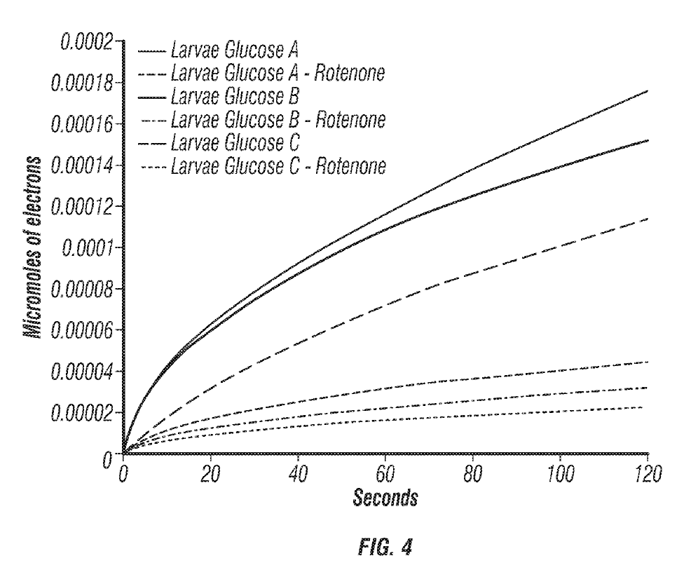

It was found that the electron flux (which constitutes part of the metabolic flux and is directly relatable to oxygen consumption or substrate turnover) generated from whole drosophilae larvae using glucose as a substrate without inhibitor present was 1.468×10⁻³ micromoles of electrons with a standard deviation of 3.115×10⁻⁴ for 120 seconds of measurement (FIG. 4).

### Example 3—Discussion

It was demonstrated that the attenuation of electron flux (which constitutes part of the metabolic flux and is directly relatable to oxygen consumption or substrate turnover) generated from mouse liver mitochondria suspension was diminished in the presence of rotenone when pyruvate is used as the metabolic substrate shown in FIG. 2. In addition, it was also found that changing the metabolic substrate, such as pyruvate to citrate which is a metabolic substrate that enters the metabolic pathway after the pyruvate decarboxylase complex which is the classical target for rotenone, did not exhibit significant inhibition shown in FIG. 3. It was also demonstrated that the attenuation of metabolic flux generated from a whole drosophilae larvae in a glucose solution was diminished in the presence of rotenone shown in FIG. 4. These types of examples demonstrate that this technology is capable of quantitatively measuring metabolic flux of many types of samples in a very short timeframe allowing for extremely high throughput measurements of metabolic flux which has not been demonstrated by any other prior art.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### E. REFERENCES

The following references, to the extent that they provide exemplary procedural or other details supplementary to those set forth herein, are specifically incorporated herein by reference.
1) U.S. Pat. No. 8,845,879
2) Schubert et al., Methods in Enzymology, vol. 137, 1988.
3) Sarma et al., Biosensors and Bioelectronics, 24, available online Oct. 18, 2008, 2313-2322.
Arechederra et al., Analyst, 2011, 135, 3747-3752.
4) Germain et al., Nitroaromatic Actuation of Mitochondrial Bioelectrocatalysis for Self-Powered Explosive Sensors; Journal of the American Chemical Society 130, 15272-15273 (2008).
4) Thomas et al., Effects of annealing on mixture-cast membranes of Nafion and quaternary ammonium bromide salts, Journal of Membrane Science 213 (2003) 55-66.
5) Moore et al. Improving the Environment for Immobilized Dehydrogenase Enzymes by Modifying Nafion with Tetraalkylammonium Bromides, Biomacromolecules 5 (2004) 1241-1247.
6) Klotzbach et al., Effects of hydrophobic modification of chitosan and Nafion on transport properties, ion-exchange capacities, and enzyme immobilization; Journal of Membrane Science 282 (2006) 276-283.
7) Moore et al., Effects of surfactants on the transport properties of redox species through Nafion membranes; Journal of Membrane Science 255 (2005) 233-238.
8) Addo et al., Evaluating Enzyme Cascades for Methanol/Air Biofuel Cells Based on NAD-Dependent Enzymes; Electroanalysis, vol. 22, No. 7-8, pp. 807-812 (2010).
9) Arechederra et al., Organelle-based biofuel cells: Immobilized mitochondria on carbon paper electrodes, Electrochimica Acta 53, 6698-6703 (2008).
10) Arechederra et al., Mitochondrial bioelectrocatalysis for biofuel cell applications; Electrochimica Acta 54, 7268-7273 (2009).
11) Mabalirajan et al., Mitochondrial Structural Changes and Dysfunction Are Associated with Experimental Allergic Asthma; The Journal of Immunology; vol. 181, 3540-3548 (2008).
12) Scatena et al., The role of mitochondria in pharmacotoxicology: a reevaluation of an old, newly emerging topic; Am. J. Physiol. 293, C12-C21, (2007).
13) Boelsterli et al., The heterozygous Sod2+/− mouse: modeling the mitochondrial role in drug toxicity, Drug Discovery Today 13, 982-988,(2008).
14) Degli Esposti et al., Natural substances (acetogenins) from the family Annonaceae are powerful inhibitors of mitochondrial NADH dehydrogenase (Complex I)Biochem. J. 301, 161-167 (1994).
15) Dykens et al., The significance of mitochondrial toxicity testing in drug development; Drug Discovery Today 12, 777-785, (2007).
16) Dykens et al., Strategies to reduce late-stage drug attrition due to mitochondrial toxicity; Expert Review of Molecular Diagnostics 7, 161-175 (2007).
17) Kovacic et al., Mechanism of Mitochondrial Uncouplers, Inhibitors, and Toxins: Focus on Electron Transfer, Free Radicals, and Structure-Activity Relationships; Current Medicinal Chemistry 12, 2601-2623 (2005).
18) Mabalirajan et al., Esculetin Restores Mitochondrial Dysfunction and Reduces Allergic Asthma Features in Experimental Murine Model; Journal of Immunology. 183, 2059-2067 (2009).
19) Nadanaciva et al., Current Concepts in Drug-Induced Mitochondrial Toxicity; Current Protocols in Toxicology 2, 1-9, (May 2009).
Petit et al., Mitochondria are sensors for HIV drugs; Trends in Pharmacological Sciences 26, 258-264 (2005).
20) Wang et al., Inhibitors of Cytochrome c Release with Therapeutic Potential for Huntington's Disease Journal of Neuroscience 28, 9473-9485 (Sep. 2008).
21) Yang et al., Apoptosis of murine lupus T cells induced by the selective cyclooxygenase-2 inhibitor celecoxib: Molecular mechanisms and therapeutic potential; International Immunopharmacology 7, 1411-1421 (2007).
22) Hatefi et al., Inhibitors and Activators of the Mitochondrial Reduced Diphosphopyridine Nucleotide Dehydrogenase; Journal of Biological Chemistry; 244, 2358-2365 (1969).
23) Wojtczak et al., Basic Mitochondrial Physiology in Cell Viability and Death, John Wiley & Sons, New York, 1-36 (2008).
24) Drose et al., Ambivalent effects of diazoxide on mitochondrial ROS production at respiratory chain complexes I and III; Biochimica et Biophysica Acta 1790 (2009) 558-565.
25) Nagao et al., Human mitochondrial carbonic anhydrase: cDNA cloning, expression, subcellular localization, and mapping to chromosome 16 Proc. Natl. Acad. Sci. USA vol. 90, 7623-7627 (Aug. 1993).
26) Nagao et al., Mitochondrial carbonic anhydrase (isozyme V) in mouse and rat:cDNA cloning, expression, subcellular localization, processing, and tissue distribution; Proc. Natl. Acad. Sci. USA vol. 91, 10330-10334 (Oct. 1994).
27) Maurer et al., Lithium-induced enhancement of mitochondrial oxidative phosphorylation in human brain tissue; Bipolar Disorders, vol. 11, pp. 515-522, 2009.

1. A method of measuring an oxidoreductive reaction in an organelle, cell or organism comprising:
(a) providing said organelle, cell or organism in a conductive solution comprising an electrolyte;
(b) locating a first electrode in said conductive solution within about 2.0 mm of said organelle, cell or organism, wherein said organelle, cell or organism may or may not be in direct contact with said first electrode;
(c) locating a second electrode in said conductive solution, wherein said organelle, cell or organism may or may not be in direct contact with said first electrode;
(d) applying a potential to said first electrode, and an opposite potential to said second electrode, thereby generating a potential gradient; and
(e) measuring electrical current across said first and second electrodes, wherein detection of said electrical current indicates the presence of an oxidoreductive reaction in said organelle, cell or organism, or the production of electrochemically active compounds by said organelle, cell or organism.

2. The method of claim 1, wherein said cell is located in a tissue sample or tissue culture.

3. The method of claim 1, wherein said organelle is a nucleolus, a nucleus, a ribosome, a vesicle, a rough endoplasmic reticulum, a Golgi apparatus, cytoskeleton, a smooth endoplasmic reticulum, a mitochondrion, a mitoplast, a vacuole, a chloroplast, a thylakoid, a lysosome, and a centriole.

4. The method of claim 1, wherein said organism is a single-cell organism, a cell line, or embryo.

5. The method of claim 1, wherein said organism is a multicellular organism.

6. The method of claim 1, wherein said multicellular organism is an invertebrate larva, invertabrate pupae, mature invertabrate, vertebrate in in all stages of development including just after embryonic stage.

7. The method of claim 1, wherein said conductive solution comprises metabolic substrates.

8. The method claim 1, wherein said first electrode is a working electrode and said second electrode is a counter electrode.

9. The method of claim 1, wherein said conductive solution is a buffered solution comprising DMSO.

10. The method of claim 1, wherein said conductive solution is a hypotonic or hypertonic solution.

11. The method of claim 1, wherein said conductive solution is an isotonic solution.

12. The method of claim 9, further comprising locating a third electrode in said conductive solution, said third electrode being a quasi-reference electrode.

13. The method claim 4, wherein said organism is rendered sufficiently permeable to allow compounds to taken up by said organism.

14. The method of claim 13, wherein said organism is intact.

15. The method of claim 13, wherein said organism has been dissected.

16. The method of claim 1, further comprising performing steps (a)-(e) a second time.

17. The method of claim 16, wherein said organelle, cell or organism has been subjected to a treatment between the first and second measuring steps.

18. The method of claim 17, wherein said treatment comprises culturing of said organelle, cell or organism with a single component or multiple of the following: a toxin, a pesticide, a herbicide, an explosive, a solvent, an industrial chemical, a pollutant, a therapeutic small molecule, a biological agent, a genetic modifying agent, a radioactive compound, signaling cell compound, an organelle signaling compound, a redox compound, a therapeutic large molecule, a drug antibody conjugate, a nanomaterial, a polymer, a surfactant, an oligosaccharide, a saccharide, a fatty compound, a hormone, a cholesterol, a cytokine, a protein, a coenzyme, a vitamin, an antioxidant, a catalyst, a DNA section, an RNA section, an extract from another organism, an acid, a base, an isotopically enriched compound, an exposure to electromagnetic radiation from any portion of the electromagnetic spectrum or exposure to electromagnetic fields, an exposure to elevated or reduced temperatures, an exposure to elevated or reduced pressures, a gaseous compound.

19. The method of claim 1, wherein said organelle, cell or organism, said first and second electrodes, and said conductive solution are disposed in a tissue culture dish, a well of a tissue culture tray, inserted into an organism, a screen printed electrode, in a test tube, or vial.

20. The method of claim 1, wherein said electrical current is quantified.
